(19) United States
(12) Patent Application Publication
(10) Pub. No.: US 2005/0177882 A1
(43) Pub. Date: Aug. 11, 2005

(54) Method and system for fusion and activation following nuclear transfer in reconstructed embryos

(76) Inventors: William G Gavin, Dudley, MA (US); David Melican, Fiskdale, MA (US); Robin E. Butler, Spencer, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/501,305

(22) PCT Filed: Jan. 8, 2003
(86) PCT No.: PCT/US03/00452

Related U.S. Application Data
(60) Provisional application No. 60/347,701, filed on Jan. 11, 2002.

(51) Int. Cl. A01K 67/027

(52) U.S. Cl. 800/14; 800/15; 800/16; 800/17

(57) ABSTRACT

The present invention provides data to demonstrate that the re-fusion, of a mammalian karyoplast to an enucleated in vivo ovulated cocyte, following an unsuccessful initial simultaneous electrical fusion and activation event offers an additional alternative and improvement in the creation of activated and fused nuclear transfer-capable embryos for the production of live off-spring in various mammalian non-human species including goats, pigs, rodents, primates, rabbits and cattle. Additionally, multiple electrical pulses offers an alternative and more efficient activation method in a simultaneous fusion and activation methodology for viable offspring production in a animal nuclear transfer program.

### FIELD OF THE INVENTION

The present invention relates to improved methods for the fusion and activation of reconstructed embryos for use in nuclear transfer procedures in non-human mammals. More specifically, the current invention provides a method to improve the activation of reconstructed embryos in nuclear transfer procedures through the use of at least two electrical activation procedures.

### BACKGROUND OF THE INVENTION

The present invention relates generally to the field of somatic cell nuclear transfer (SCNT) and to the creation of desirable transgenic animals. More particularly, it concerns methods for generating somatic cell-derived cell lines, transforming these cell lines, and using these transformed cells and cell lines to generate transgenic non-human mammalian animal species.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, are time-consuming, costly and unreliable. Moreover, these processes are completely incapable of allowing a specific animal line from producing gene products, such as desirable protein therapeutics that are otherwise entirely absent from the genetic complement of the species in question (i.e., spider silk proteins in bovine milk).

The development of technology capable of generating transgenic animals provides a means for exceptional precision in the production of animals that are engineered to carry specific traits or are designed to express certain proteins or other molecular compounds. That is, transgenic animals are animals that carry a gene that has been deliberately introduced into somatic and/or germline cells at an early stage of development. As the animals develop and grow the protein product or specific developmental change engineered into the animal becomes apparent.

At present the techniques available for the generation of transgenic domestic animals are inefficient and time-consuming typically producing a very low percentage of viable embryos. During the development of a transgene, DNA sequences are typically inserted at random, which can cause a variety of problems. The first of these problems is insertional inactivation, which is inactivation of an essential gene due to disruption of the coding or regulatory sequences by the incoming DNA. Another problem is that the transgene may either be not incorporated at all, or incorporated but not expressed. A further problem is the possibility of inaccurate regulation due to positional effects. This refers to the variability in the level of gene expression and the accuracy of gene regulation between different founder animals produced with the same transgenic constructs. Thus, it is not uncommon to generate a large number of founder animals and often confirm that less than 5% express the transgene in a manner that warrants the maintenance of the transgenic line.

Additionally, the efficiency of generating transgenic domestic animals is low, with efficiencies of 1 in 100 offspring generated being transgenic not uncommon (Wall, 1997). As a result the cost associated with generation of transgenic animals can be as much as 250-500 thousand dollars per expressing animal (Wall, 1997).

Prior art methods have typically used embryonic cell types in cloning procedures. This includes work by Campbell et al (Nature, 1996) and Stice et al (Biol. Reprod., 1996). In both of those studies, embryonic cell lines were derived from embryos of less than 10 days of gestation. In both studies, the cells were maintained on a feeder layer to prevent overt differentiation of the donor cell to be used in the cloning procedure. The present invention uses differentiated cells. It is considered that embryonic cell types could also be used in the methods of the current invention along with cloned embryos starting with differentiated donor nuclei.

Thus, according to the present invention, multiplication of superior genotypes of mammals, including caprines, is possible. This will allow the multiplication of adult animals with proven genetic superiority or other desirable traits. Progress will be accelerated, for example, in many important mammalian species including goats, rodents, cows and rabbits. By the present invention, there are potentially billions of fetal or adult cells that can be harvested and used in the cloning procedure. This will potentially result in many identical offspring in a short period.

Thus although transgenic animals have been produced by various methods in several different species, methods to readily and reproducibly produce transgenic animals capable of expressing the desired protein in high quantity or demonstrating the genetic change caused by the insertion of the transgene(s) at reasonable costs are still lacking.

Accordingly, a need exists for improved methods of nuclear transfer that will allow an increase in production efficiencies in the development of transgenic animals, particularly with regard to the activation of fused cells during the simultaneous fusion and activation of cell couplets in an effort to produce viable transgenic offspring more reliably and efficiently.

### SUMMARY OF THE INVENTION

Briefly stated, the current invention provides a method for cloning a non-human mammal through a nuclear transfer process comprising: obtaining desired differentiated mammalian cells to be used as a source of donor nuclei; obtaining at least one oocyte from a mammal of the same species as the cells which are the source of donor nuclei; enucleating the at least one oocyte; transferring the desired differentiated cell or cell nucleus into the enucleated oocyte; simultaneously fusing and activating the cell couplet to form a first transgenic embryo; activating a cell-couplet that does not fuse to create a first transgenic embryo but that is activated after an initial electrical shock by providing at least one additional activation protocol including an additional electrical shock to form a second transgenic embryo; culturing the activated first and/or second transgenic embryo(es) until greater than the 2-cell developmental stage; and finally transferring the first and/or second transgenic embryo into a suitable host mammal such that the embryo develops into a fetus. Typically, the above method is completed through the use of a donor cell nuclei in which a desired gene has been inserted, removed or modified prior to insertion of said differentiated mammalian cell or cell nucleus into said enucleated oocyte. Also of note is the fact that the oocytes used are preferably matured in vitro prior to enucleation.

Moreover, the method of the current invention also provides for optimizing the generation of transgenic animals through the use of caprine oocytes, arrested at the Metaphase-II stage, that were enucleated and fused with donor somatic cells and simultaneously activated. Analysis of the milk of one of the transgenic cloned animals showed high-level production of human of the desired target transgenic protein product.

It is also important to point out that the present invention can also be used to increase the availability of CICM cells, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimericism can be used to avoid immunological rejection among animals of the same species as well as between species.

### BRIEF DESCRIPTION OF THE DRAWINGS

FIG. 1 Shows A Generalized Diagram of the Process of Creating Cloned Animals through Nuclear Transfer.

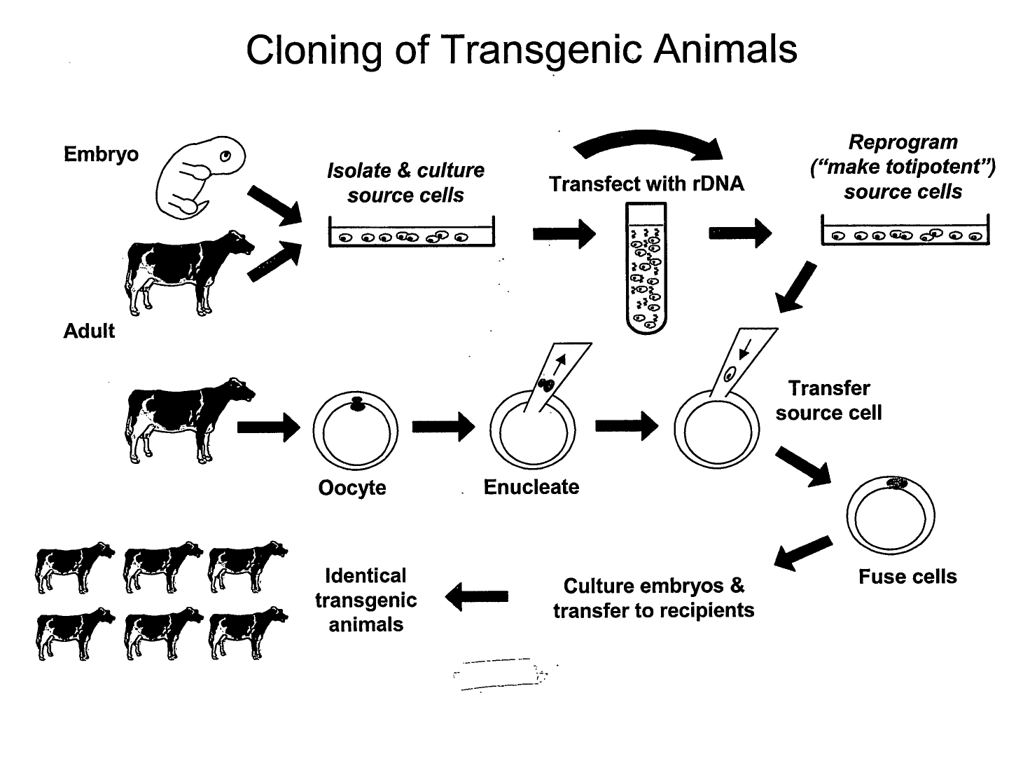

### DESCRIPTION OF THE PREFERRED EMBODIMENT

The following abbreviations have designated meanings in the specification:

Abbreviation Key:
Somatic Cell Nuclear Transfer (SCNT)
Cultured Inner Cell Mass Cells (CICM)
Nuclear Transfer (NT)
Synthetic Oviductal Fluid (SOF)
Fetal Bovine Serum (FBS)
Polymerase Chain Reaction (PCR)
Bovine Serum Albumin (BSA)

Explanation of Terms:
Caprine—Of or relating to various species of goats.
Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.
Fusion Slide—A glass slide for parallel electrodes that are placed a fixed distance apart. Cell couplets are placed between the electrodes to receive an electrical current for fusion and activation.
Cell Couplet—An enucleated oocyte and a somatic or fetal karyoplast prior to fusion and/or activation.
Cytocholasin-B—A metabolic product of certain fungi that selectively and reversibly blocks cytokinesis while not effecting karyokinesis.
Cytoplast—The cytoplasmic substance of eukaryotic cells.
Karyoplast—A cell nucleus, obtained from the cell by enucleation, surrounded by a narrow rim of cytoplasm and a plasma membrane.
Somatic Cell—Any cell of the body of an organism except the germ cells.
Parthenogenic—The development of an embryo from an oocyte without the penetrance of sperm
Transgenic Organism—An organism into which genetic material from another organism has been experimentally transferred, so that the host acquires the genetic traits of the transferred genes in its chromosomal composition.
Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

The present invention relates to a system for an increasing the number of transgenic embryos developed for nuclear transfer procedures. The current invention provides an improved method for the creation of fused and activated embryos, following an unsuccessful initial simultaneous electrical fusion and activation event. This capability offers an improvement in the efficiency of the creation of activated and fused nuclear transfer-capable embryos for the production of live offspring in various mammalian non-human species including goats, pigs, rodents, primates, rabbits and cattle.

In addition, the present invention relates to cloning procedures in which cell nuclei derived from differentiated fetal or adult mammalian cells, which include non-serum starved differentiated fetal or adult caprine cells, are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce chimeric embryos, fetuses and/or offspring.

Fusion of a donor karyoplast to an enucleated cytoplast, and subsequent activation of the resulting couplet are important steps required to successfully generate live offspring by somatic cell nuclear transfer. Electrical fusion of a donor karyoplast to a cytoplast is the most common method used. More importantly however, several methods of activation, and the timing of the activation steps, used in nuclear transfer methodologies to initiate the process of embryo development in numerous livestock species have been published. In mammals, while there are species differences, the initial signaling events and subsequent Ca⁺² oscillations induced by sperm at fertilization are the normal processes that result in oocyte activation and embryonic development (Fissore et al., 1992 and Alberio et al., 2001). Both chemical and electrical methods of Ca⁺² mobilization are currently utilized to activate couplets generated by somatic cell nuclear transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al., Biol. Reprod. 2001; Lai et al., 2001; Yong et al., 1998; and Kasinathan et al., Nature Biotech. 2001). However, there is quite a large degree of variability in the sequence, timing and methodology used for fusion and activation.

Prior art techniques rely on the use of blastomeres of early embryos for nuclear transfer procedure. This approach is limited by the small numbers of available embryonic blastomeres and by the inability to introduce foreign genetic material into such cells. In contrast, the discoveries that differentiated embryonic, fetal, or adult somatic cells can function as karyoplast donors for nuclear transfer have provided a wide range of possibilities for germline modification. According to the current invention, the use of recombinant somatic cell lines for nuclear transfer, and improving this procedures efficiency by increasing the number of available cells through the use of “reconstructed” embryos, not only allows the introduction of transgenes by traditional transfection methods into more transgenic animals but also increases the efficiency of transgenic animal production substantially while overcoming the problem of founder mosaicism.

We have previously shown that simultaneous electrical fusion and activation can successfully produce live offspring in the caprine species, and other animals. In our current experiments, we investigated the use of additional electrical activation events, following initial successful simultaneous electrical fusion and activation, to more closely mimic sperm-induced Ca⁺² oscillations and generate both embryos and live offspring by somatic cell nuclear transfer. Finally, we determined the ability of re-fusing donor karyoplasts to enucleated cytoplasts, which did not successfully fuse at the initial simultaneous electrical fusion and activation event, to generate both goat embryos and live offspring by somatic cell nuclear transfer.

The data underlying the instant invention demonstrates that a single additional electrical activation event following the initial successful simultaneous electrical fusion and activation is more efficient, compared to simultaneous electrical fusion and activation alone in the ability to produce a live offspring. In subsequent experiments, we expanded the experimental protocol to include both a single or timed multiple additional electrical activation event following the initial successful simultaneous electrical fusion treatment. The results of the subsequent experiments demonstrate that while different numbers of additional electrical activation steps are comparable in the ability to generate nuclear transfer embryos capable of establishing pregnancies at day 55 of gestation, both methods were more efficient than the experiments. Bondolli et al., have previously reported that additional electrical activation events can successfully generate live offspring by nuclear transfer in the porcine species. Other reports (Collas et al., 1993) demonstrate that additional electrical activation events can successfully generate parthenogenetic embryos in the bovine species. Our results here suggest that additional electrical activation following the initial successful simultaneous electrical fusion and activation of a goat karyoplast and enucleated in vivo ovulated oocyte in a separate protocol methodology may offer an alternative and more efficient method of activation using nuclear transfer in various animals, in particular the caprine species.

The efficiency of electrical fusion of a karyoplast to an enucleated cytoplast varies based on species and the cell type used. However, in our experience with the goat, and as reported by others (Baguisi et al., 1999; and Stice et al., 1992), there is a sub-population of couplets that do not successfully fuse during the initial fusion attempt. In these experiments, we determined the ability of an additional re-fusion attempt following an unsuccessful initial simultaneous electrical fusion and activation event to generate both goat embryos and live offspring by somatic cell nuclear transfer. In experiments, the data demonstrates that re-fusion was both capable and more efficient, compared to simultaneous electrical fusion and activation alone (Baguisi et al., 1999), or a single additional electrical activation event following the initial successful simultaneous electrical fusion and activation, in the ability to produce live offspring. In subsequent experiments, we confirmed our observations that re-fusion of non-fused couplets were able to generate nuclear transfer embryos capable of establishing pregnancies at day 55 of gestation.

Donor karyoplasts were obtained from a primary fetal somatic cell line derived from a 40-day transgenic female fetus produced by artificial insemination of a negative adult female with semen from a transgenic male. Live offspring were produced with two nuclear transfer procedures. In one protocol, caprine oocytes at the arrested Metaphase-II stage were enucleated, electrofused with donor somatic cells and simultaneously activated. In the second protocol, activated in vivo caprine oocytes were enucleated at the Telophase-II stage, electrofused with donor karyoplasts and simultaneously activated a second time to induce genome reactivation. Three healthy identical female offspring were born. Genotypic analyses confirmed that all cloned offspring were derived from the donor cell line. Analysis of the milk of one of the transgenic cloned animals showed high-level production of human anti-thrombin III, similar to the parental transgenic line. Thus, through the methodology and system employed in the current invention transgenic animals, goats, were generated by somatic cell nuclear transfer and were shown to be capable of producing a target therapeutic protein in the milk of a cloned animal.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Wilmut et al., and Campbell et al., reported using a single electrical pulse for fusion of the reconstructed embryo followed by a delay for a number of hours prior to activation of the embryo chemically. Other reports have demonstrated the different electrical and chemical stimuli that could be used for activation in various species (Koo et al., 2000; and Fissore A., et al.,). The current invention provides for the use of somatic cell nuclear transfer by simultaneous fusion and activation with no delay involved between the two events, with the use of subsequent additional electrical pulses to an activated and fused embryo. Subsequent investigation into fusion and activation techniques has led to alternative methodology provided in the current invention disclosure that provide improved efficiencies and make the process of producing transgenic animals or cell lines more reliable and efficient.

In the process of developing the current methodology to increase the low efficiency of fused and activated embryo's available through the prior art an investigation was performed to evaluate how to utilize reconstructed embryos that do not fuse initially but have been activated. Thereafter experiments were performed to look at multiple electrical pulses in a test species (e.g., goats). The same methodology was also tested in the porcine model for oocyte activation following nuclear transfer and for live piglet production. This was performed to better mimic what is seen in vivo when a sperm normally penetrates and fertilizes an oocyte and induces calcium oscillations (Alberio et al., 2001; and Ducibella et al., 1998).

Materials and Methods

Estrus synchronization and superovulation of donor does used as oocyte donors, and micro-manipulation was performed as described in Gavin W. G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Goats.

The herds of pure- and mixed-breed scrapie-free Alpine, Saanen and Toggenburg dairy goats used for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses produced by artificially inseminating 2 non-transgenic female animals with fresh-collected semen from a transgenic male animal. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I. U. each/ml)], and were cultured in 25 cm² flasks. A confluent monolayer of primary fetal cells was harvested by trypsinization after 4 days of incubation and then maintained in culture or cryopreserved.

Sexing and Genotyping of Donor Cell Lines.

Genomic DNA was isolated from fetal tissue, and analyzed by polymerase chain reaction (PCR) for the presence of a target signal sequence, as well as, for sequences useful for sexing. The target transgenic sequence was detected by amplification of a 367-bp sequence. Sexing was performed using a zfX/zfY primer pair and Sac I restriction enzyme digest of the amplified fragments.

Preparation of Donor Cells for Embryo Reconstruction.

A transgenic female line (CFF6) was used for all nuclear transfer procedures. Fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 7 days. On the 7th day following the first addition of low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) 1 to 3 hours prior to fusion to the enucleated oocytes.

Oocyte Collection.

Oocyte donor does were synchronized and superovulated as previously described (Gavin W. G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I. U. each/ml).

Cytoplast Preparation and Enucleation.

Oocytes with attached cumulus cells were discarded. Cumulus-free oocytes were divided into two groups: arrested Metaphase-II (one polar body) and Telophase-II protocols (no clearly visible polar body or presence of a partially extruding second polar body). The oocytes in the arrested Metaphase-II protocol were enucleated first. The oocytes allocated to the activated Telophase-II protocols were prepared by culturing for 2 to 4 hours in M199/10% FBS. After this period, all activated oocytes (presence of a partially extruded second polar body) were grouped as culture-induced, calcium-activated Telophase-II oocytes (Telophase-II-Ca) and enucleated. Oocytes that had not activated during the culture period were subsequently incubated 5 minutes in M199, 10% FBS containing 7% ethanol to induce activation and then cultured in M199 with 10% FBS for an additional 3 hours to reach Telophase-II (Telophase-II-EtOH protocol).

All oocytes were treated with cytochalasin-B (Sigma, 5 μg/ml in M199 with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. Telophase-II-Ca and Telophase-II-EtOH oocytes were enucleated by removing the first polar body and the surrounding cytoplasm (10 to 30% of cytoplasm) containing the partially extruding second polar body. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in M199 for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Starting at between 10 to 90 minutes post-fusion, most preferably at 30 minutes post-fusion, the presence of an actual karyoplast/cytoplast fusion is determined. For the purposes of the current invention fused couplets may receive an additional activation treatment (double pulse). This additional pulse can vary in terms of voltage strength from 0.1 to 5.0 kV/cm for a time range from 10 to 80 μsec. Preferably however, the fused couplets would receive an additional single electrical pulse (double pulse) of 0.4 or 2.0 kV/cm for 20 μsec. The delivery of the additional pulse could be initiated at least 15 minutes hour after the first pulse, most preferably however, this additional pulse would start at 30 minutes to 2 hours following the initial fusion and activation treatment to facilitate additional activation. In the other experiments, non-fused couplets were re-fused with a single electrical pulse. The range of voltage and time for this additional pulse could vary from 1.0 kV/cm to 5.0 kV/cm for at least 10 μsec occurring at least 15 minutes following an initial fusion pulse. More preferably however, the additional electrical pulse varied from of 2.2 to 3.2 kV/cm for 20 μsec starting at 30 minutes to 1 hour following the initial fusion and activation treatment to facilitate fusion. All fused and fusion treated couplets were returned to SOF/FBS plus 5 μg/ml cytochalasin-B. The couplets were incubated at least 20 minutes, preferably 30 minutes, at 37-39° C. in a humidified gas chamber containing approximately 5% CO₂ in air.

An additional version of the current method of the invention provides for an additional single electrical pulse (double pulse), preferably of 2.0 kV/cm for the cell couplets, for at least 20 μsec starting at least 15 minutes, preferably 30 minutes to 1 hour, following the initial fusion and activation treatment to facilitate additional activation. The voltage range for this additional activation pulse could be varied from 1.0 to 6.0 kV/cm.

Alternatively, in subsequent efforts the remaining fused couplets received at least three additional single electrical pulses (quad pulse) most preferably at 2.0 kV/cm for 20 μsec, at 15 to 30 minute intervals, starting at least 30 minutes following the initial fusion and activation treatment to facilitate additional activation. However, it should be noted that in this additional protocol the voltage range for this additional activation pulse could be varied from 1.0 to 6.0 kV/cm, the time duration could vary from 10 μsec to 60 μsec, and the initiation could be as short as 15 minutes or as long as 4 hours following initial fusion treatments. In the subsequent experiments, non-fused couplets were re-fused with a single electrical pulse of 2.6 to 3.2 kV/cm for 20 μsec starting at 1 hours following the initial fusion and activation treatment to facilitate fusion. All fused and fusion treated couplets were returned to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air for at least 30 minutes. Mannitol can be used to substitute for Cytocholasin-B.

Starting at 30 minutes following re-fusion, the success of karyoplast/cytoplast re-fusion was determined. Fusion treated couplets were washed with equilibrated SOF/FBS, then transferred to equilibrated SOF/FBS plus 5 μg/ml cycloheximide. The couplets were incubated at 37-39° C. in a humidified gas chamber containing approximately 5% CO₂ in air for up to 4 hours.

Following cycloheximide treatment, couplets were washed extensively with equilibrated SOF medium supplemented with at least 0.1% bovine serum albumin, preferably at least 0.7%, preferably 0.8%, plus 100 U/ml penicillin and 100 μg/ml streptomycin (SOF/BSA). Couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

Nuclear Transfer Embryo Culture and Transfer to Recipients.

All nuclear transfer embryos were co-cultured on monolayers of primary goat oviduct epithelial cells in 50 μl droplets of M199 with 10% FBS overlaid with mineral oil. Embryo cultures were maintained in a humidified 39° C. incubator with 5% CO₂ for 48 hours before transfer of the embryos to recipient does. Recipient embryo transfer was performed as previously described ²².

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2α (Lutalyse, Upjohn). Parturition occurred within 24 hours after treatment. Kids were removed from the dam immediately after birth, and received heat-treated colostrum within 1 hour after delivery.

Genotyping of Cloned Animals.

Shortly after birth, blood samples and ear skin biopsies were obtained from the cloned female animals (e.g., goats) and the surrogate dams for genomic DNA isolation. Each sample was first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKem®, ME) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with α-³²P dCTP using the Prime-It® kit (Stratagene, La Jolla, Calif.). Hybridization was executed at 65° C. overnight. The blot was washed with 0.2×SSC, 0.1% SDS and exposed to X-OMAT™ AR film for 48 hours.

Milk Protein Analyses.

Hormonal induction of lactation for the juvenile female transgenic animals was performed at two months-of-age. The animals were hand-milked once daily to collect milk samples for hAT expression analyses. Western blot and rhAT activity analyses were performed as described (Edmunds, T. et al., 1998).

In the experiments performed during the development of the current invention, following enucleation and reconstruction, the karyoplast/cytoplast couplets were incubated in equilibrated Synthetic Oviductal Fluid medium supplemented with 1% to 15% fetal bovine serum, preferably at 10% FBS, plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

Results

As summarized in Table 1, in the experiments, of 1646 couplets in which the initial single simultaneous fusion and activation pulse was attempted, 114 couplets lysed and 720 couplets fused (43.7%). Of the 720 fused couplets, 364 fused couplets received the double pulse, 13 couplets lysed and 351 double-pulsed couplets were cultured. A total of 812 couplets from the initial fusion attempt, which did not fuse, were re-fused. From these re-fusion attempts 54 couplets lysed and 346 couplets fused (42.6%). The overall fusion rate for both the initial fusion and re-fusion was 1066 couplets fused (64.8%) of 1646 couplets in which fusion was attempted.

TABLE 1
Nuclear transfer fusion analysis

| | # Couplets fused/ |
|---|---|
| | # Couplets treated |
| Fusion type | (%) |
| Single pulse | 720/1646 (43.7) |
| Re-fuse | 346/812 (42.6) |

Table 2 summarizes results of term pregnancies for surrogate recipient does receiving nuclear transfer embryos based on fusion and activation type. In these experiments 4 recipient does (6.1%) that received embryos generated from re-fused couplets produced term pregnancies, while 1 recipient doe (2.7%) that received embryos generated from double pulsed couplets produced a term pregnancy. Alternatively, none of the does that received embryos generated from single pulsed couplets produced term pregnancies in experimental animals.

TABLE 2
Nuclear transfer pregnancy analysis

| | # Term recipients/ |
|---|---|
| | # Recipients |
| Fusion type | (%) |
| Single pulse | 0/35 |
| Double pulse | 1/37 (2.7) |
| Re-fuse | 4/66 (6.1) |

The results of offspring produced based on fusion and activation type is summarized in Table 3. In the experiments 4 offspring (1.2%) were produced from 346 fusion positive couplets generated by re-fusion, while 1 offspring (0.3%) was produced from 351 fusion positive couplets generated by the double pulse method of activation. Alternatively, no offspring were produced from 353 fusion positive couplets generated from simultaneous fusion and activation.

TABLE 3
Nuclear transfer fusion and activation offspring analysis

| | | # Offspring |
|---|---|---|
| Fusion type | # Fused couplets | (%) |
| Single pulse | 353 | 0 |
| Double pulse | 351 | 1 (0.3) |
| Re-fuse | 346 | 4 (1.2) |

Table 4 summarizes the results of the production effort for the development of transgenic founder animals, in this set of experiments the animals produced were goats. However, the techniques presented herein are also useful in other mammalian species. This data represents the period of May and June 2001. While this table details the production effort, the most relevant aspects are the numbers of reconstructed couplets that successfully fused and the resulting number of developing embryos that were transferred to recipient does. A total of 902 embryos generated by somatic cell nuclear transfer were transplanted to 138 surrogate recipient does, and five recipient does (3.6%) produced term pregnancies yielding 5 healthy offspring.

TABLE 4
Nuclear transfer data 2000/2001 season (Apr. 30-Jun. 22, 2001)

| | | |
|---|---|---|
| # Donors | 178 | |
| # Ovulations/Donor | 18.1 | (3213 total ovulations) |
| # Ova/Donor | 11.2 | (1998 total ova) |
| # Enucleated | 1951 | (97.6% oocytes recovered) |
| # Reconstructed | 1784 | (91.4% oocytes enucleated) |
| # Couplets fusion attempted | 1646 | (92.3% oocytes reconstructed) |
| # Couplets fused | 1066 | (64.8% fusion attempted) |
| # Cleaved | 536 | (50.3% couplets fused) |
| # Recipients | 138 | (902 total transferred) |
| # Embryos/Recipient | 6.5 | (range 1-24) |
| # Pregnancies | 5/138 | (3.6%) |
| # Offspring | 5 | |

Based on the results of these experiments, in the subsequent experiments no fused single pulsed couplets were transferred to recipient does. Alternatively, all fused couplets were double or quad pulse treated. In addition, re-fusion of non-fused couplets was performed in all subsequent experiments. As summarized in Table 5, in the subsequent experiments, of 2599 couplets in which the initial single simultaneous fusion and activation pulse was attempted, 85 couplets lysed and 1404 couplets fused (54.0%). Of the 1404 fused couplets, 825 fused couplets received the double pulse, 22 couplets lysed and 803 double-pulsed couplets were cultured. Of the remaining fused couplets, 579 fused couplets received the quad pulse, 57 couplets lysed and 522 quad-pulsed couplets were cultured. A total of 1110 couplets from the initial fusion attempt, which did not fuse, were re-fused. From these re-fusion attempts, 33 couplets lysed and 672 couplets fused (60.5%). The overall fusion rate for both the initial fusion and re-fusion was 2076 couplets fused (79.9%) of 2599 couplets in which fusion was attempted.

TABLE 5
Nuclear transfer fusion analysis

| | # Couplets fused/ |
|---|---|
| | # Couplets treated |
| Fusion type | (%) |
| Single pulse | 1404/2599 (54.0) |
| Re-fuse | 672/1110 (60.5) |

Table 6 summarizes the results of Day 55 ultrasounds for surrogate recipient does receiving nuclear transfer embryos based on fusion and activation type. In these experiments 7 recipient does (13.2%) that received embryos generated from double pulsed couplets were pregnant at Day 55 of gestation. Alternatively, 4 recipient does (8.5%) that received embryos generated from quad pulsed couplets and 4 recipient does (4.6%) that received embryos generated from re-fused couplets were pregnant at Day 55 of gestation.

TABLE 6
Nuclear transfer pregnancy analysis

| | # Recipients pregnant/ |
|---|---|
| | # Recipients |
| Fusion Type | Day 55 gestation (%) |
| Double pulse | 7/53 (13.2) |
| Quad pulse | 4/47 (8.5) |
| Re-fuse | 4/87 (4.6) |

In the current example, goats were used as the transgenic animals. Therefore ultrasounds of pregnant does were taken on day 55 of their gestation period. The results of Day 55 ultrasounds based on fusion and activation type is summarized in Table 7. In the subsequent experiments 9 fetuses (1.1%) were developing from 803 fusion positive couplets generated by the double pulse method of activation, while 6 fetuses (1.1%) were developing from 522 fusion positive couplets generated by the quad pulse method of activation. Alternatively, 5 fetuses (0.7%) were developing from 672 fusion positive couplets generated by re-fusion.

TABLE 7
Nuclear transfer fusion and activation offspring analysis

| | | # Fetuses |
|---|---|---|
| | | Day 55 |
| | # Fused | gestation |
| Fusion type | couplets | (% fused) |
| Double pulse | 803 | 9 (1.1) |
| Quad pulse | 522 | 6 (1.1) |
| Re-fuse | 672 | 5 (0.7) |

Table 8 summarizes the results of the production effort for the development of transgenic founder animals. This subsequent data represents the period of September 2001 through December 2001. While this table details the production effort, the most relevant aspects are the numbers of reconstructed couplets that successfully fused and the resulting number of developing embryos that were transferred to recipient does. A total of 1562 embryos generated by somatic cell nuclear transfer were transplanted to 262 surrogate recipient does. Day 55 ultrasounds have been performed on 188 recipients, with 15 confirmed pregnancies (8.0%) displaying fetal development.

TABLE 8
Nuclear transfer data 2001/2002 season (Aug. 27-Dec. 21, 2001)

| | | |
|---|---|---|
| Total Ovulations | 5266 | |
| # Donors | 381 | |
| Ovulations/Donor | 13.8 | |
| # Ova Retrieved | 2965 | (56% of ovulations) |
| # Ova/Donor | 7.8 | |
| # Ova ovulated & aspirated | 3188 | |
| # enucleated | 3001 | (94% oocytes recovered) |
| # reconstructed | 2798 | (93% oocytes enucleated) |
| # couplets fusion attempted | 2599 | (93% oocytes reconstructed) |
| # couplets fused | 2076 | (80% fusion attempted) |
| # cleaved | 765 | (40% couplets fused) |
| | | (57% at approx. 48 hrs) |
| # nuclear transfer embryos | 1562 | |
| transferred | | |
| # Recipients | 262 | |
| # Embryos/Recipient | 6.0 | (range 1-14) |
| # Pregnancies | 15/188 | (8.0%) |
| # Offspring | NA | |

The present invention allows for increased efficiency of transgenic procedures by providing for an additional generation of activated and fused transgenic embryos. These embryos can be implanted in a surrogate animal or can be clonally propagated and stored or utilized. Also by combining nuclear transfer with the ability to modify and select for these cells in vitro, this procedure is more efficient than previous transgenic embryo techniques. According to the present invention, these transgenic cloned embryos can be used to produce CICM cell lines or other embryonic cell lines. Therefore, the present invention eliminates the need to derive and maintain in vitro an undifferentiated cell line that is conducive to genetic engineering techniques.

Thus, in one aspect, the present invention provides a method for cloning a mammal. In general, a mammal can be produced by a nuclear transfer process comprising the following steps:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a first transgenic embryo;
(vi) activating a cell-couplet that does not fuse to create a first transgenic embryo but that is activated after an initial electrical shock by providing at least one additional activation protocol including an additional electrical shock to form a second transgenic embryo;
(vii) culturing said activated first and/or second transgenic embryo until greater than the 2-cell developmental stage; and
(viii) transferring said first and/or second transgenic embryo into a host mammal such that the embryo develops into a fetus.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and offspring of those mammals. The present invention is preferably used for cloning caprines. The present invention further provides for the use of nuclear transfer fetuses and nuclear transfer and chimeric offspring in the area of cell, tissue and organ transplantation.

In another aspect, the present invention provides a method for producing CICM cells. The method comprises:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a first transgenic embryo;
(vi) activating a cell-couplet that does not fuse to create a first transgenic embryo but that is activated after an initial electrical shock by providing at least one additional activation protocol including an additional electrical shock to form a second transgenic embryo;
(vii) culturing said activated first and/or second transgenic embryo until greater than the 2-cell developmental stage; and
(viii) culturing cells obtained from said cultured activated embryo to obtain CICM cells.

Also CICM cells derived from the methods described herein are advantageously used in the area of cell, tissue and organ transplantation, or in the production of fetuses or offspring, including transgenic fetuses or offspring. Differentiated mammalian cells are those cells, which are past the early embryonic stage. Differentiated cells may be derived from ectoderm, mesoderm or endoderm tissues or cell layers.

An alternative method can also be used, one in which the cell couplet can be exposed to multiple electrical shocks to enhance fusion and activation. In general, the mammal will be produced by a nuclear transfer process comprising the following steps:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
employing at least two electrical shocks to a cell-couplet to initiate fusion and activation of said cell-couplet into an activated and fused embryo.
(vii) culturing said activated and fused embryo until greater than the 2-cell developmental stage; and
(viii) transferring said first and/or second transgenic embryo into a host mammal such that the embryo develops into a fetus;
wherein the second of said at least two electrical shocks is administered at least 15 minutes after an initial electrical shock.

Mammalian cells, including human cells, may be obtained by well-known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures. Again the present invention is novel because differentiated cell types are used. The present invention is advantageous because the cells can be easily propagated, genetically modified and selected in vitro.

Suitable mammalian sources for oocytes include goats, sheep, cows, pigs, rabbits, guinea pigs, mice, hamsters, rats, primates, etc. Preferably, the oocytes will be obtained from caprines and ungulates, and most preferably goats. Methods for isolation of oocytes are well known in the art. Essentially, this will comprise isolating oocytes from the ovaries or reproductive tract of a mammal, e.g., a goat. A readily available source of goat oocytes is from hormonal induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields).

The stage of maturation of the oocyte at enucleation and nuclear transfer has been reported to be significant to the success of nuclear transfer methods. (First and Prather 1991). In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially goats, the oocyte activation period generally occurs at the time of sperm contact and penetrance into the oocyte plasma membrane.

After a fixed time maturation period, which ranges from about 10 to 40 hours, and preferably about 16-18 hours, the oocytes will be enucleated. Prior to enucleation the oocytes will preferably be removed and placed in EMCARE media containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in EMCARE media, preferably containing 7.5 micrograms per milliliter cytochalasin B, for immediate enucleation, or may be placed in a suitable medium, for example an embryo culture medium such as CR1aa, plus 10% FBS, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in EMCARE or SOF, and then viewing the oocytes under ultraviolet irradiation for less than 10 seconds. The oocytes that have been successfully enucleated can then be placed in a suitable culture medium.

In the present invention, the recipient oocytes will preferably be enucleated at a time ranging from about 10 hours to about 40 hours after the initiation of in vitro or in vivo maturation, more preferably from about 16 hours to about 24 hours after initiation of in vitro or in vivo maturation, and most preferably about 16-18 hours after initiation of in vitro or in vivo maturation.

A single mammalian cell of the same species as the enucleated oocyte will then be transferred into the perivitelline space of the enucleated oocyte used to produce the activated embryo. The mammalian cell and the enucleated oocyte will be used to produce activated embryos according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Ponimaskin et al., 2000).

Also, in some cases (e.g. with small donor nuclei) it may be preferable to inject the nucleus directly into the oocyte rather than using electroporation fusion. Such techniques are disclosed in Collas and Barnes, Mol. Reprod. Dev., 38: 264-267 (1994), incorporated by reference in its entirety herein.

The activated embryo may be activated by known methods. Such methods include, e.g., culturing the activated embryo at sub-physiological temperature, in essence by applying a cold, or actually cool temperature shock to the activated embryo. This may be most conveniently done by culturing the activated embryo at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may best be effected by simultaneously, although protocols for sequential activation do exist. In terms of activation the following cellular events occur:
(i) increasing levels of divalent cations in the oocyte, and
(ii) reducing phosphorylation of cellular proteins in the oocyte.

The above events can be exogenously stimulated to occur by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation may be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

Accordingly, it is to be understood that the embodiments of the invention herein providing for an increased availability of activated and fused “reconstructed embryos” are merely illustrative of the application of the principles of the invention. It will be evident from the foregoing description that changes in the form, methods of use, and applications of the elements of the disclosed method for the improved use of reconstructed embryos for SCNT are novel and may be modified and/or resorted to without departing from the spirit of the invention, or the scope of the appended claims.

### LITERATURE CITED AND INCORPORATED BY REFERENCE

1. Alberio R, et al., Mammalian Oocyte Activation: Lessons from the Sperm and Implications for Nuclear Transfer, INT J DEV BIOL 2001; 45: 797-809.
2. Alberio R, et al., Remodeling of Donor Nuclei, DNA Synthesis, and Ploidy of Bovine Cumulus Cell Nuclear Transfer Embryos: Effect of Activation Protocol, MOL REPROD DEV 2001; 59: 371-379.
3. Baguisi A, et al., Production of Goats by Somatic Cell Nuclear Transfer, NAT BIOTECH 1999; 17: 456461.
4. Booth P J, et al., Effect of Two Activation Treatments and Age of Blastomere Karyoplasts on In Vitro Development of Bovine Nuclear Transfer Embryos, MOL REPROD DEV 2001; 60: 377-383.
5. Bondioli K, et al., Cloned Pigs from Cultured Skin Fibroblasts Derived from A H-Transferase Transgenic Boar, MOL REPROD DEV 2001; 60: 189-195.
6. Bondioli K R, Westhusin M E And C R Loony, Production of Identical Bovine Offspring by Nuclear Transfer, THERIOGENOLOGY 1990; 33: 165-174.
7. Campbell, K H S, Mcwhire J., Ritchie W A And I. Wilmut. Sheep Cloned by Nuclear Transfer From a Cultured Cell Line, NATURE 1996; 380: 64-66.
8. Cibelli J B, et al., Cloned Transgenic Calves Produced From Nonquiescent Fetal Fibroblasts. SCIENCE 1998; 280: 1256-1258.
9. Collas P, and Barnes F L., Nuclear Transplantation by Microinjection of Inner Cell Mass and Granulosa Cell Nuclei, MOL REPROD DEV. 1994 July; 38(3): 264-7.
10. Collas P. Electrically Induced Calcium Elevation, Activation, and Parthenogenic Development of Bovine Oocytes. MOL REPROD 1993; 34: 212-223.
11. Ducibella T., Biochemical and Cellular Insights Into the Temporal Window of Normal Fertilization, THERIO 1998: 49: 53-65.
12. Edmunds, T. et al., Transgenically Produced Human Antithrombin—Structural and Functional Comparison to Human Plasma-Derived Antithrombin, BLOOD 1998; 91:4561-4571.
13. First N L, and Prather R S, Genomic Potential in Mammals, DIFFERENTIATION 1991 September; 48(1):1-8.
14. Fissore A, et al., Patterns of Intracellular Ca²⁺ Concentrations in Fertilized Bovine Eggs, BIOL REPROD 1992; 47: 960-969.
15. Gavin, W. G., Gene Transfer Into Goat Embryos, TRANSGENIC ANIMALS—GENERATION AND USE, L. M. Houdebine ed., (Harwood Academic Publishers Gmbh., 1996).
16. Groupen C G, et al., Activation of In Vivo and In Vitro Derived Porcine Oocytes by Using Multiple Electrical Pulses, REPROD FERT DEV 1999; 11: 457462.
17. Kasinathan P, et al., Effect of Fibroblast Donor Cell Age and Cell Cycle on Development of Bovine Nuclear Transfer Embryos In Vitro, BIOL REPROD 2001; 64(5): 1487-1493.
18. Kasinathan P, et al., Production of Calves from G1 Fibroblasts, NATURE BIOTECH 2001; 19: 1176-1178.
19. Kato Y. et al., Cloning of Calves from Various Somatic Cell Types of Male and Female Adult, Newborn and Fetal Cows, J REPROD FERT 2000; 120: 231-237.
20. Koo D B, et al., In Vitro Development Of Reconstructed Porcine Oocytes After Somatic Cell Nuclear Transfer. BIOL REPROD 2000; 63: 986-992.
21. Lai, L, et al., Feasibility of producing Porcine Nuclear Transfer Embryos by Using G2/M-Stage Fetal Fibroblasts as Donors, BIOL REPROD 2001; 65: 1558-1564.
22. Meng L, Ely J J, Stouffer R L And D P Wolf, Rhesus Monkeys Produced by Nuclear Transfer, BIOL REPROD 1997; 57: 454-459.
23. Park K W, et al., Developmental Potential of Porcine Nuclear Transfer Embryos Derived from Transgenic Fibroblasts Infected with the Gene for the Green Fluorescent Protein: Comparison of Different Fusion/Activation Conditions, BIOL REPROD 2001; 65: 1681-1685.
24. Polejaeva I A, et al., Cloned Pigs Produced by Nuclear Transfer from Adult Somatic Cells, NATURE 2000: 407: 86-90.
25. Ponimaskin E, et al., Sendai Virosomes Revisited: Reconstitution with Exogenous Lipids Leads to Potent Vehicles for Gene Transfer, VIROLOGY, 2000 April. 10; 269(2): 391-403.
26. Stice S L, et al., Pluripotent Bovine Embryonic Cell Lines Direct Embryonic Development Following Nuclear Transfer, BIOL REPROD. 1996 January; 54(1): 100-10.
27. Stice S L and J M Robl, Nuclear Reprogramming in Nuclear Transplant Rabbit Embryo, BIOL REPROD 1988; 39(3): 657-64.
28. Wakayama T, et al., Full Term Development of Mice from on Enucleated Oocytes Injected with Cumulus Cell Nuclei, NATURE 1998; 394: 369-374.
29. Wall R J, et al., Transgenic Dairy Cattle: Genetic Engineering on a Large Scale, J DAIRY SCI. 1997 September; 80(9): 2213-24.
30. Wells D N, Misica P M And H R Tervit, Production of Cloned Calves Following Nuclear Transfer with Cultured Adult Mural Granulosa Cells, Biol Reprod 1999; 60: 996-1005.
31. Willadsen S M, Nuclear Transplantation in Sheep Embryos, NATURE 1986; 320: 63-65.
32. Wilmut I, et al., Viable Offspring Derived From Fetal and Adult Mammalian Cells. NATURE 1997; 385: 810-813.
33. Yang X, S Jiang, A Kovacs and R H Foote, Nuclear Totipotency of Cultured Rabbit Morulae to Support Full-Term Development Following Nuclear Transfer, BIOL REPROD 1992; 47: 636-643.
34. Yong Z And L Yuqiang, Nuclear-Cytoplasmic Interaction and Development of Goat Embryos Reconstructed by Nuclear Transplantation: Production of Goats by Serially Cloning Embryos, BIOL REPROD 1998; 58: 266-269.
35. Zou X, et al., Production of Cloned Goats from Enucleated Oocytes Injected with Cumulus Cell Nuclei or Fused with Cumulus Cells, CLONING 2001; 3 (1): 31-37.

1. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) activating a cell-couplet that does not fuse to create a first transgenic embryo but that is activated after an initial electrical shock by providing at least one additional activation protocol including an additional electrical shock to form a second transgenic embryo;
(vii) culturing said activated first and/or second transgenic embryo(es) until greater than the 2-cell developmental stage; and
(viii) transferring said first and/or second transgenic embryo into a host mammal such that the embryo develops into a fetus.

2. The method of claim 1, wherein said donor differentiated mammalian cell to be used as a source of donor nuclei or donor cell nucleus is from mesoderm.

3. The method of claim 1, wherein said donor differentiated mammalian cell to be used as a source of donor nuclei or donor cell nucleus is from endoderm.

4. The method of claim 1, wherein said donor differentiated mammalian cell to be used as a source of donor nuclei or donor cell nucleus is from ectoderm.

5. The method of claim 1, wherein said donor differentiated mammalian cell to be used as a source of donor nuclei or donor cell nucleus is from fetal somatic tissue.

6. The method of claim 1, wherein said donor differentiated mammalian cell to be used as a source of donor nuclei or donor cell nucleus is from fetal somatic cells.

7. The method of claim 1, wherein said donor differentiated mammalian cell to be used as a source of donor nuclei or donor cell nucleus is from a fibroblast.

8. The method of claim 1, wherein said donor differentiated mammalian cell to be used as a source of donor nuclei or donor cell nucleus is from an ungulate.

9. The method of either claims 1 or 8, wherein said donor cell or donor cell nucleus is from an ungulate selected from the group consisting of bovine, ovine, porcine, equine, caprine and buffalo.

10. The method of claim 1, wherein said donor differentiated mammalian cell to be used as a source of donor nuclei or donor cell nucleus is from an adult non-human mammalian somatic cell.

11. The method of claim 1, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

12. The method of claim 1, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

13. The method of claim 1, wherein said at least one oocyte is matured in vivo prior to enucleation.

14. The method of claim 1, wherein said at least one oocyte is matured in vitro prior to enucleation.

15. The method of claim 1, wherein said non-human mammal is a rodent.

16. The method of claim 1, wherein said donor differentiated mammalian cell to be used as a source of donor nuclei or donor cell nucleus is a non-quiescent somatic cell or a nucleus isolated from said non-quiescent somatic cell.

17. The method of either claims 1 or 8, wherein the fetus develops into an offspring.

18. The method of claim 1, wherein said at least one oocyte is enucleated about 10 to 60 hours after initiation of in vitro maturation.

19. The method of claim 1, wherein a desired gene is inserted, removed or modified in said differentiated mammalian cell or cell nucleus prior to insertion of said differentiated mammalian cell or cell nucleus into said enucleated oocyte.

20. The resultant offspring of the methods of claims 1 or 19.

21. The resultant offspring of claim 19 further comprising wherein the offspring created as a result of said nuclear transfer procedure is chimeric.

22. The method of claim 1, wherein cytocholasin-B is used in the cloning protocol.

23. The method of claim 1, wherein cytocholasin-B is not used in the cloning protocol.

24. A method for producing cultured inner cell mass cells, comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) activating a cell-couplet that does not fuse to create a first transgenic embryo but that is activated after an initial electrical shock by providing at least one additional activation protocol including an additional electrical shock to form a second transgenic embryo; and
(vi) culturing cells obtained from said cultured activated embryo to obtain cultured inner cell mass cells.

25. The method of claim 24, wherein said donor differentiated mammalian cell to be used as a source of donor nuclei or donor cell nucleus is from mesoderm.

26. The method of claim 24, wherein said donor differentiated mammalian cell to be used as a source of donor nuclei or donor cell nucleus is from endoderm.

27. The method of claim 24, wherein said donor differentiated mammalian cell to be used as a source of donor nuclei or donor cell nucleus is from ectoderm.

28. The method of claim 24, wherein said donor differentiated mammalian cell to be used as a source of donor nuclei or donor cell nucleus is from fetal somatic tissue.

29. The method of claim 24, wherein said donor differentiated mammalian cell to be used as a source of donor nuclei or donor cell nucleus is from fetal somatic cells.

30. The method of claim 24, wherein said donor differentiated mammalian cell to be used as a source of donor nuclei or donor cell nucleus is from a fibroblast.

31. The method of claim 24, wherein said donor differentiated mammalian cell to be used as a source of donor nuclei or donor cell nucleus is from an ungulate.

32. The method of either claims 24 or 31, wherein said donor cell or donor cell nucleus is from an ungulate selected from the group consisting of bovine, ovine, porcine, equine, caprine and buffalo.

33. The method of claim 24, wherein said donor differentiated mammalian cell to be used as a source of donor nuclei or donor cell nucleus is from an adult mammalian somatic cell.

34. The method of claim 24, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

35. The method of claim 24, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

36. The method of claim 24, wherein said at least one oocyte is matured in vivo prior to enucleation.

37. The method of claim 24, wherein said at least one oocyte is matured in vitro prior to enucleation.

38. The method of claim 24, wherein said mammalian cell is derived from a rodent.

39. The method of claim 24, wherein said donor differentiated mammalian cell to be used as a source of donor nuclei or donor cell nucleus is a non-quiescent somatic cell or a nucleus isolated from said non-quiescent somatic cell.

40. The method of either claims 24 or 31, wherein any of said cultured inner cell mass cells fetus develops into a non-human offspring.

41. The method of claim 24, wherein said at least one oocyte is enucleated about 10 to 60 hours after initiation of in vitro maturation.

42. The method of claim 24, wherein a desired gene is inserted, removed or modified in said differentiated mammalian cell or cell nucleus prior to insertion of said differentiated mammalian cell or cell nucleus into said enucleated oocyte.

43. The resultant offspring of the methods of claims 24 or 42.

44. The resultant offspring of claim 42 further comprising wherein any non-human offspring created as a result of said nuclear transfer procedure is chimeric.

45. The method of claim 24, wherein cytocholasin-B is used in the protocol.

46. The method of claim 24, wherein cytocholasin-B is not used in the protocol.

47. The method of claim 24, wherein cytocholasin-B is used in the protocol.

48. The method of claim 24, wherein said cultured inner cell mass cells are used to develop a functional organ for transplantation.

49. The method of claim 24, wherein said cultured inner cell mass cells are used in organogenesis.

50. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said oocytes;
(iv) transferring the desired differentiated cell or cell nucleus into the enucleated oocyte;
employing at least two electrical shocks to a cell-couplet to initiate fusion and activation of said cell-couplet into an activated and fused embryo.
(vii) culturing said activated and fused embryo until greater than the 2-cell developmental stage;
(viii) transferring said first and/or second transgenic embryo into a host mammal such that the embryo develops into a fetus;
wherein the second of said at least two electrical shocks is administered at least 15 minutes after an initial electrical shock; and
wherein a desired gene is inserted, removed or modified in said differentiated mammalian cell or cell nucleus prior to insertion of said differentiated mammalian cell or cell nucleus into said enucleated oocyte.
